(19) United States
(12) Patent Application Publication
(10) Pub. No.: US 2005/0277623 A1
(43) Pub. Date: Dec. 15, 2005

(54) Treatment for averting or delaying premature delivery

(76) Inventor: Hartmut M Hanauske-Abel, Edge Water, NJ (US)

Correspondence Address: Michael L Goldman;Nixon Peabody, Clinton Square, P O Box 31051, Rochester, NY, 14603, US

(21) Appl. No.: 10/498,748

(22) PCT Filed: Dec. 18, 2002
(86) PCT No.: PCT/US02/41111

§ 371 Date: Jul. 11, 2005

Related U.S. Application Data
(60) Provisional application No. 60/344,253, filed on Dec. 20, 2001.

(51) Int. Cl. A61K 31/65; A61K 31/195

(52) U.S. Cl. 514/152; 514/566

(57) ABSTRACT

The present invention relates to a method for averting or delaying premature delivery of mammalian infants. This method includes administering to a pregnant mammalian female an agent which interferes with degradation of cervical and/or fetal membrane connective tissue elements under conditions effective to avert or delay premature delivery of an infant by the female.

This application claims benefit of U.S. Provisional patent Application Ser. No. 60/344,253, filed Dec. 20, 2001.

### FIELD OF THE INVENTION

The present invention relates to a treatment for averting or delaying premature delivery by administering an agent which interferes with the degradation of cervical and/or fetal membrane connective tissue.

### BACKGROUND OF THE INVENTION

Premature delivery, or preterm birth (birth at <37 completed weeks of gestation), is the second leading cause of neonatal and infant mortality, only surpassed by birth defects (1). In up to 40% of premature deliveries, the immediate cause is the preterm premature rupture of fetal membranes (2). The short and long-term care for premature neonates, the products of preterm births, is an extraordinary effort in medical and economic terms. At present, premature neonates of 30 weeks gestational age comprise 1.4% of annual life births, yet consume 32.1% of total neonatal care costs, or $3.3 billion, expenses related not to mortality, but to duration of treatment in the neonatal intensive care unit (NICU) (3). A significant fraction—in some studies every second baby—of the premature neonates that eventually graduate from the NICU, will survive only at the expense of serious disabilities that are the price for life-saving NICU treatment and that may affect every major organ system. These handicapped pediatric patients incur long-term costs estimated at $6 billion annually (4). For these reasons, medical means of averting or delaying premature delivery, or molecular concepts to identify and develop such means, have significant and immediate practical consequences.

In order for delivery to occur, the cervical segment of the female uterus, which retains the human conceptus, has to ‘soften’ so as to allow passage of the baby out of the uterine cavity. Increased fragility of the fetal membranes with subsequent rupture is also a decisive, early birth event. Both cervical softening and rupture of membranes, as well as several other signs and symptoms, are the clinical representations of major, rapid changes in the molecular composition of the connective tissue of these anatomical structures. These changes invariably involve the degradation of connective tissue elements, resulting in a marked ‘loosening’ of tensile strength, and are mediated by lytic enzymes, especially the matrix metalloproteinases (MMPs), of which several dozen are known at present.

The causal involvement of certain MMPs in specific obstetrical events has been established. For instance, amniotic MMP-8 is significantly increased by microbial invasion of the uterine cavity and is involved in infection-triggered preterm premature rupture of membranes (5); elevated amniotic MMP-1 concentrations are associated with preterm premature rupture of membranes both in the presence and the absence of infection (6). In fact, the human amniochorion expresses a multitude of these matrix-degrading enzymes and has a fully functional network of MMPs (7). MMP-9, which is specialized in degrading the collagenous elements of basement membranes, displays a particularly immediate involvement with preterm premature rupture of membranes: Preterm labor with intact membranes leading to preterm delivery in the absence of infection is highly associated with a significant increase in the median concentration of the active forms of MMP-9, but not of the active forms of MMP-2; and preterm rupture of membranes is highly associated with a significant increase in the concentration of the active forms of MMP-9 and with a significant decrease in the active forms of MMP-2 (8). It is also established that distinct and specific molecular pathways exist for MMP mediation of particular obstetrical events. Thus, quantitative polymerase chain reaction results demonstrate an increased mRNA expression for MMP-2 and MMP-9 in prematurely ruptured membranes compared with preterm labor membranes; and at the protein level, enzyme-linked immunosorbent assays document increases in the amniotic fluid concentrations of immunoreactive and bioactive MMP-2 and MMP-9 in fluids obtained from women with premature rupture of membranes when compared to women with preterm labor (9). In summary, while other MMPs are also involved in the preterm premature rupture of fetal membranes, it is MMP-9 that has been consistently identified as causally involved [e.g. 2, 10-14].

Under physiological conditions, the lytic and matrix-degrading activity of MMPs is held in check by at least three different, endogenous tissue inhibitors of metalloproteinases (TIMPs). Preterm premature rupture of fetal membranes is characterized by a marked amniotic imbalance between MMPs and TIMPs (9,15), the relative and/or absolute amount of TIMPs being significantly decreased. This imbalance is particularly prominent for MMP-9: TIMP-free forms of active MMP-9 were only observed in the amniotic fluid of women who had suffered preterm premature rupture of fetal membranes (16).

The present invention is directed to overcoming these deficiencies in the art.

### SUMMARY OF THE INVENTION

The present invention relates to a method for averting or delaying premature delivery of mammalian infants. This method includes administering to a pregnant mammalian female an agent which interferes with degradation of cervical and/or fetal membrane connective tissue elements under conditions effective to avert or delay premature delivery of an infant by the female.

The present invention involves the use of exogenous agents that restore the amniotic balance between MMPs and MMP inhibitors, by decreasing MMP production and/or by directly interacting with and blocking MMPs. The ensuing reduction in bioactivity, in particular of MMP-9, allows their clinical use with the therapeutic intention of averting or delaying premature delivery as the result of preterm premature rupture of fetal membranes.

The present invention achieves a number of benefits. Firstly, it teaches the systemic/topical use of drugs, with a human toxicity profile established in adults and neonates (i.e. the tetracyclines), for averting or delaying preterm, premature rupture of fetal membranes and thus premature delivery, a serious, expensive, and potentially life-threatening medical condition. Secondly, the present invention identifies the molecular targets involved (i.e. the MMPs and in particular MMP-9) and provides a strategy to correct the amniotic imbalance between MMPs and their endogenous inhibitors, by compensatory administration of tetracyclines as exogenous small-molecule MMPs inhibitors. The strategy of the present invention suggests the usefulness of other small-molecule MMPs inhibitors, such as chemically modified non-antimicrobial tetracyclines or chelating agents directed against the catalytic zinc atom of the MMPs.

### DETAILED DESCRIPTION OF THE INVENTION

The present invention relates to a method for averting or delaying premature delivery of mammalian infants. This method includes administering to a pregnant mammalian female an agent which interferes with degradation of cervical and/or fetal membrane connective tissue elements under conditions effective to avert or delay premature delivery of an infant by the female.

Metalloproteinase inhibitors, small-molecule exogenous agents that inhibit MMPs, are now being developed and tested clinically for their special effect as anti-tumor agents (17). Their utility in diseases not obviously related to cancer, metastasis, and angiogenesis is only now emerging, and a few initial studies in non-oncological conditions have explored potential benefits, e.g. in arthritic diseases (18).

For use during pregnancy to avert or delay premature delivery as the result of preterm premature rupture of fetal membranes, any small-molecule exogenous agents that inhibit MMPs must not only effectively inhibit MMP bioactivity in vivo, especially the one of MMP-9, but must also be non-teratogenic in humans.

Surprisingly, we have discovered that the Tetracyclines meet both preconditions. Clinically employed Tetracyclines with a good safety record, such as minocycline and doxycycline, have been identified as inhibitors of, in particular, MMP-9 bioactivity under in vivo conditions when administered per routine dosage (19-22). Tetracyclines also lack teratogenic risk when administered to the mother even during the period of fetal organogenesis, i.e. in the first trimester (e.g., ref. 23).

However, the clinical use of Tetracyclines during pregnancy is not apparent, and in fact is routinely not recommended by standard textbooks (e.g. 24) and not part of state-of-the-art care. Tetracyclines accumulate in the developing calcified structures of bone and teeth due to their ability to bind calcium. While their depressive effect on bone is temporary and entirely reversible due to tissue remodeling, the deciduous teeth assume a brown discoloration and the permanent teeth can display hypoplasia of the enamel. In light of the grave, long-term potential sequelae that preterm premature rupture of fetal membranes imposes on newborns, e.g. severe neurological damage, such cosmetic effects classify as minor consequences. Thus, the use of tetracyclines in pregnancy to prevent preterm premature rupture of fetal membranes is entirely unexpected.

Administration of Tetracyclines may occur topically or orally once an appropriate diagnosis has been reached, e.g. by measurement of established markers for preterm delivery (25). High-dose vaginal delivery of tetracyclines may also have a suppressive effect on the microbial population causing local inflammation, generally considered a factor predisposing to preterm delivery. Of note, tetracyclines are known to increase the biosynthesis of fibrillar collagens (26, 27). Thus, with a single compound, FDA-approved for use in humans, a synergistic profile can be achieved which is highly relevant for this major subset of premature deliveries : i) metalloproteinase inhibition, especially of MMP-9; ii) antimicrobial activity; iii) profibrotic effect. Systemic application and local delivery might be combined to achieve optimal biological effect.

The method of the present invention can be carried out with any mammal, but, of course, is particularly useful in treating a human. It can be used to avert and/or delay premature delivery before 37 weeks gestational age.

Besides tetracylines, metal chelators, metalloproteinase inhibitors, and combinations thereof can be used to carry out the present invention.

Useful Tetracyclines include minocycline, doxycyclin, metastat, chemically modified derivatives thereof, or combinations thereof.

A suitable metal chelator is a zinc chelator, such as a hydroxypyridinone. The hydroxypyridinone can be deferiprone, a chemically modified derivative thereof, or combinations thereof.

Useful metalloproteinase inhibitors include marimastat, prinomastat, solimastat, neovastat, carboxyamido-triazole, BMS-275291, chemically modified derivatives thereof, or combinations thereof.

Although preferred embodiments have been depicted and described in detail herein, it will be apparent to those skilled in the relevant art that various modifications, additions, substitutions and the like can be made without departing from the spirit of the invention and these are therefore considered to be within the scope of the invention as defined in the claims which follow.

### REFERENCES

The following references (cited above) are hereby incorporated by reference in their entirely:
1. Peters etal., “Deaths: Final Data for 1996. National Vital Statistics Reports,” Hyattsville, Md.: National Center for Health Statistics 47(9):83 (1998).
2. Athayde et al., “A Role for Matrix Metalloproteinase-9 in Spontaneous Rupture of the Fetal Membranes,” Am J Obstet Gynecol 179:1248-1253 (1998).
3. St. John et al., “Costs of Neonatal Care According to Gestational Age at Birth and Survival Status,” Am J Obstet Gynecol 182:170-175 (2000).
4. Zupancic et al., “Economics of Prematurity in the Era of Managed Care,” Clin Perinatol 27(2):483-497 (2000).
5. Maymon et al., “Human Neutrophil Collagenase (Matrix Metalloproteinase 8) in Parturition, Premature Rupture of the Membranes, and Intrauterine Infection,” Am J Obstet Gynecol 183:94-99 (2000).
6. Maymon et al., “Evidence for the Participation of Interstitial Collagenase (Matrix Metalloproteinase 1) in Preterm Premature Rupture of Membranes,” Am J Obstet Gynecol 183:914-20 (2000).
7. Fortunato et al., “Screening of Novel Matrix Metalloproteinases (MMPs) in Human Fetal Membranes,” J Assist Reprod Genet 19:483-486 (2002).
8. Maymon et al., “Evidence of In Vivo Differential Bioavailability of the Active Forms of Matrix Metalloproteinases 9 and 2 in Parturition, Spontaneous Rupture of Membranes, and Intra-amniotic Infection,” Am J Obstet Gynecol 183:887-894 (2000).
9. Fortunato et al., “Distinct Molecular Events Suggest Different Pathways for Preterm Labor and Premature Rupture of Membranes,” Am J Obstet Gynecol 184:1399-1405 (2001).
10. Tsatas et al., “Differential Expression of Proteases in Human Gestational Tissues Before, During and After Spontaneous-onset Labour at Term,” J Reprod Fertil 116:43-49 (1999).
11. Romero et al., “Fetal Plasma MMP-9 Concentrations are Elevated in Preterm Premature Rupture of the Membrane,” Am J Obstet Gynecol 187:1125-1130 (2002).
12. Arechavaleta-Velasco et al., “Association of Type II Apoptosis and 92-kDa Type IV Collagenase Expression in Human Amniochorion in Prematurely Ruptured Membranes with Tumor Necrosis Factor Receptor-1 Expression,” J Soc Gynecol Investig 9:60-7 (2002).
13. Agrez et al., “Matrix Metalloproteinase 9 Activity in Urine of Patients at Risk for Premature Delivery,” Am J Obstet Gynecol 181:387-388 (1999).
14. Athayde et al., “Matrix Metalloproteinases-9 in Preterm and Term Human Parturition,” J Matern Fetal Med 8:213-219 (1999).
15. Maymon et al., “A Role for the 72 kDa Gelatinase (MMP-2) and its Inhibitor (TIMP-2) in Human Parturition, Premature Rupture of Membranes and Intraamniotic Infection,” J Perinat Med 29:308-316 (2001).
16. Fortunato et al., “MMP/TIMP Imbalance in Amniotic Fluid During PROM: An Indirect Support for Endogenous Pathway to Membrane Rupture,” J Perinat Med 27:362-368 (1999).
17. Wojtowicz-Praga et al., “Matrix Metalloproteinase Inhibitors,” Invest New Drugs 15:61-75 (1997).
18. Mengshol et al., “Matrix Metalloproteinases as Therapeutic Targets in Arthritic Diseases: Bull's-eye or Missing the Mark?,” Arthritis Rheum 46:13-20 (2002).
19. Brundula et al., “Targeting Leukocyte MMPs and Transmigration: Minocycline as a Potential Therapy for Multiple Sclerosis,” Brain 125:1297-308 (2002).
20. Roach et al., Up-regulation of MMP-2 and MMP-9 Leads to Degradation of Type IV Collagen During Skeletal Muscle Reperfusion Injury; Protection by the MMP Inhibitor, Doxycycline,” Eur J Vasc Endovasc Surg 23:260-9 (2002).
21. Baxter et al., “Prolonged Administration of Doxycycline in Patients with Small Asymptomatic Abdominal Aortic Aneurysms: Report of a Prospective (Phase II) Multicenter Study,” J Vasc Surg 36:1-12 (2002).
22. Bendeck et al., “Doxycycline Modulates Smooth Muscle Cell Growth, Migration, and Matrix Remodeling After Arterial Injury,” Am J Pathol 160:1089-95 (2002).
23. Aselton et al., “First-trimester Drug Use and Congenital Disorders,” Obstet Gynecol 65:451-5 (1985).
24. Niebyl JR, “Drugs in Pregnancy and Lactation,” In: Gabbe S. G, Simpson L. J. Niebyl J. R. (eds) Obstetrics-Normal and problem pregnancies. 299-328. Churchill, N. Y. 1991.
25. Lockwood et al., “Markers of Risk for Preterm Delivery,” J Perinat Med 27:5-20 (1999). 26. Sasaki et al., “Tetracycline Administration Increases Protein (Presumably Procollagen) Synthesis and Secretion in Periodontal Ligament Fibroblasts of Streptozotocin-induced Diabetic Rats,” J Periodontal Res 27:631-9 (1992).
27. Sasaki et al., “Tetracycline Administration Increases Collagen Synthesis in Osteoblasts of Streptozotocin-induced Diabetic Rats: A Quantitative Autoraiographic Study,” Calcif Tissue Int 50:411-9 (1992).

1. A method for averting or delaying premature delivery of mammalian infants, said method comprising:
administering to a pregnant mammalian female an agent which interferes with degradation of cervical and/or fetal membrane connective tissue elements under conditions effective to avert or delay premature delivery of an infant by the female.

2. The method according to claim 1, wherein the mammal is a human.

3. The method according to claim 2, wherein said administering is carried out systemically.

4. The method according to claim 2, wherein said administering is carried out topically.

5. The method according to claim 2, wherein the agent is selected from the group consisting of tetracylines, metal chelators, metalloproteinase inhibitors, and combinations thereof.

6. The method according to claim 5, wherein the agent is a tetracycline.

7. The method according to claim 6, wherein the tetracycline is selected from the group consisting of minocycline, doxycyclin, metastat, chemically modified derivatives thereof, and combinations thereof.

8. The method according to claim 5, wherein the agent is a metal chelator.

9. The method according to claim 8, wherein the metal chelator is a zinc chelator.

10. The method according to claim 9, wherein the zinc chelator is hydroxypyridinone.

11. The method according to claim 10, wherein the hydroxypyridinone is deferiprone, a chemically modified derivative thereof, and combinations thereof

12. The method according to claim 5, wherein the agent is a metalloproteinase inhibitor.

13. The method according to claim 12, wherein the metalloproteinase inhibitor is selected from the group consisting of marimastat, prinomastat, solimastat, neovastat, carboxyamido-triazole, BMS-275291, chemically modified derivatives thereof, and combinations thereof.

14. The method according to claim 2, wherein premature delivery is birth before 37 weeks gestational age.
